(19) United States
(12) Patent Application Publication
(10) Pub. No.: US 2002/0069875 A1
(43) Pub. Date: Jun. 13, 2002

(54) Slip-proof ball-like pump for a breathing apparatus

(76) Inventor: Tzong-Fuh Kuo, Hsin Juang City (TW)

Correspondence Address: Keith Kline, PRO-TECHTOR INTERNATIONAL SERVICES, 20775 Norada Court, Saratoga, CA, 95070-3018, US

(21) Appl. No.: 09/734,935

(22) Filed: Dec. 11, 2000

(51) Int. Cl. A61M 16/00

(52) U.S. Cl. 128/202.28; 128/205.13

(57) ABSTRACT

A slip-proof ball-like pump adapted to a breathing apparatus has multiple securing devices formed on the outer periphery of the ball-like pump. The multiple securing devices enable the user to hold the ball-like pump firmly without worrying that a slip will happen during the holding to the ball.

### BACKGROUND OF THE INVENTION

1. Field of the Invention

The present invention relates to a slip-proof ball-like pump, and more particularly to a slip-proof ball-like pump for a breathing apparatus. The slip-proof ball-like pump has at least one slip-proof device formed on an outer periphery of the ball, such that even the hands of the paramedic are wet, a firm grip to the ball can still be achieved.

2. Prior Art Description

A conventional breathing apparatus is shown in FIG. 3. The breathing apparatus has a breathing mask (300), a first pipe (200) secured to the breathing mask (300) and being in communication with the breathing mask (300), a ball-like pump (100) secured to a free end of the first pipe (200) and being in communication with the first pipe (200), a second pipe (400) one end of which is secured to the ball-like pump (100) and being in communication with the ball-like pump (100) and an air bag (500) secured to the other end of the second pipe (400) and being in communication with the second pipe (400).

When this type of breathing apparatus is used in an emergency by the paramedic, the paramedic uses one hand to squeeze the ball-like pump (100) to force the air inside the air bag (500) into the nose or mouth of the person in danger. However, in most emergency cases in a rainy day, the hands of the paramedic are not easy to keep dry or they always wear gloves to protect infection. During such a situation, the paramedic are not able to hold the ball-like pump (100) firmly and a slip might happen when holding the ball-like pump (100), which is quite inconvenient to the paramedic. What is more important is that during this critical moment, seconds are vital to the person in danger. Therefore, should a slip happen during a restoration operation, a life may just slip away just because the paramedic can not have a firm grip to that ball-like pump (100).

In order to overcome the aforementioned shortcoming, an improved product is introduced into the market. The improved breathing apparatus has a ball like pump (600) with little dots formed on the outer periphery of the pump to increase the friction when hold by the user. However, this kind of design can not be hold firmly by female paramedic because the sizes of their hands are generally too small to have a firm grip.

The present invention provides an improved ball-like pump for the breathing apparatus to overcome the above mentioned shortcomings.

### SUMMARY OF THE INVENTION

The primary objective of the invention is to provide an improved ball-like pump for a breathing apparatus. The ball-like pump has at least one slip-proof device formed on the outer periphery of the ball, such that even the hands of the paramedic are wet, the paramedic can still have a firm grip to the ball so that the paramedic are able to prevent any incidents from happening due to the slippery holding to the ball.

Other objects, advantages and novel features of the invention will become more apparent from the following detailed description when taken in conjunction with the accompanying drawings.

### BRIEF DESCRIPTION OF THE DRAWINGS

FIG. 1 is a perspective view showing the slip-proof ball-like pump incorporated with a breathing apparatus and constructed in accordance with the present invention;

FIG. 2 is a perspective view showing another preferred embodiment of the invention;

FIG. 3 is a perspective view showing a conventional ball incorporated with a breathing apparatus; and

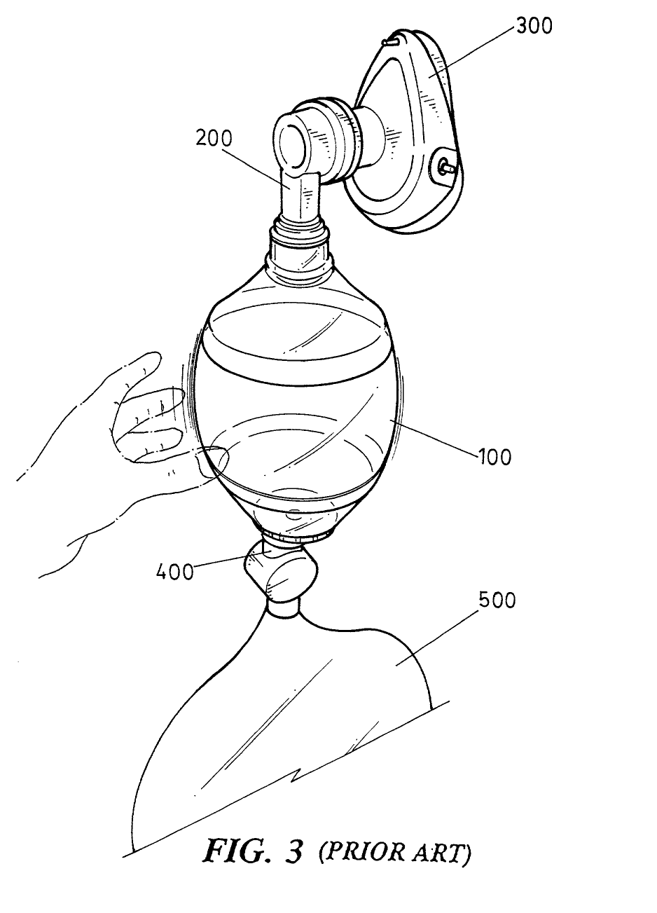

FIG. 4 is still a perspective view showing a conventional ball incorporated with the breathing apparatus.

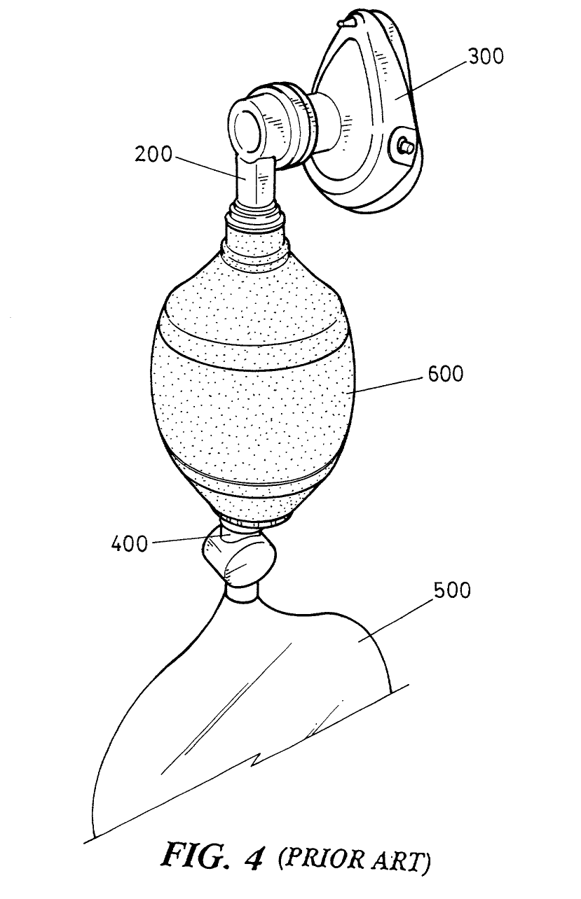

### DETAILED DESCRIPTION TO THE PREFERRED EMBODIMENT

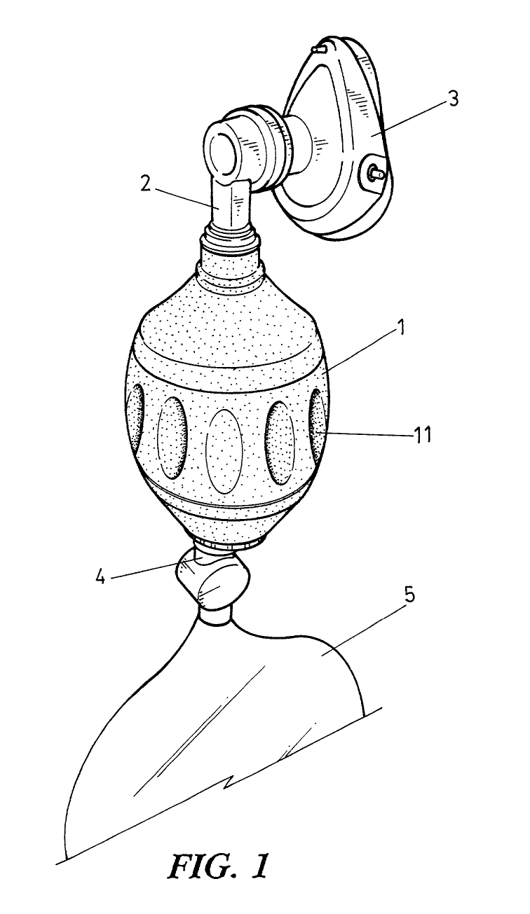

A breathing apparatus is shown in FIG. 1. The breathing apparatus has a breathing mask (3), a first pipe (2) secured to the breathing mask (3) and being in communication with the breathing mask (3), a ball-like pump (1) secured to a free end of the first pipe (2) and being in communication with the first pipe (2), a second pipe (4) one end of which is secured to the ball-like pump (1) and being in communication with the ball-like pump (1) and an air bag (5) secured to the other end of the second pipe (4) and being in communication with the second pipe (4). The previous description is the same as that described in the background. Thus, the operation thereof is omitted.

Multiple elliptical recesses (11) are defined in the outer periphery of the ball-like pump (1) such that when the paramedic are holding the ball-like pump (1), the fingers of the paramedic are able to place inside the recesses (11) to reach a firm grip to the ball-like pump (1).

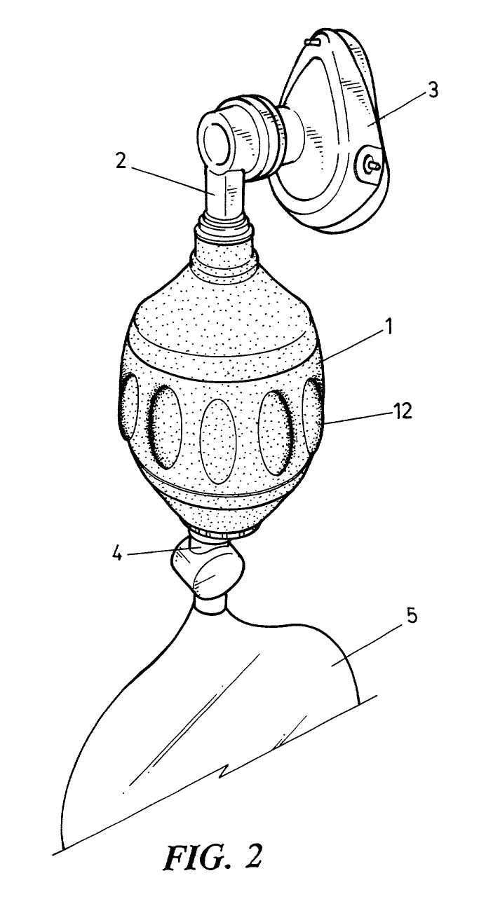

With reference to FIG. 2, another preferred embodiment of the invention is shown, wherein multiple protrusions (12) are defined in the outer periphery of the ball-like pump (1), such that when the paramedic are holding the ball-like pump (1), the fingers of the paramedic are able to clamp the protrusions (12) to have a firm grip to the ball-like pump (1). In both embodiments, the recesses (11) or the protrusions (12) are formed in line on the outer periphery of the ball-like pump (1).

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A breathing apparatus comprising:
a breathing mask;
a first pipe secured to the breathing mask and being in communication with the breathing mask;
a ball-like pump secured to a free end of the first pipe and being in communication with the first pipe and having multiple recesses defined in an outer periphery of the ball-like pump;
a second pipe one end of which is secured to the ball-like pump and being in communication with the ball-like pump; and
an air bag secured to the other end of the second pipe and being in communication with the second pipe.

2. The breathing apparatus as claimed in claim 1, wherein the multiple recesses are formed around an outer periphery of the ball-like pump.

3. A breathing apparatus comprising:
a breathing mask;
a first pipe secured to the breathing mask and being in communication with the breathing mask;
a ball-like pump secured to a free end of the first pipe and being in communication with the first pipe and having multiple protrusions formed on an outer periphery of the ball-like pump;
a second pipe one end of which is secured to the ball-like pump and being in communication with the ball-like pump; and
an air bag secured to the other end of the second pipe and being in communication with the second pipe.

4. The breathing apparatus as claimed in claim 1, wherein the multiple protrusions are formed around an outer periphery of the ball-like pump.
